(19) United States
(12) Patent Application Publication
(10) Pub. No.: US 2012/0158040 A1
(43) Pub. Date: Jun. 21, 2012

(54) Soft Tissue Tech Instrument

(76) Inventor: Jonathan Edward Dehors, Apex, NC (US)

(21) Appl. No.: 13/328,450

(22) Filed: Dec. 16, 2011

Related U.S. Application Data
(60) Provisional application No. 61/425,248, filed on Dec. 21, 2010.

(51) Int. Cl. A61B 17/00

(52) U.S. Cl. 606/201

(57) ABSTRACT

A pair of soft tissue instruments which provide a combination edge profile allowing a single tool to break up fibrous adhesions and massage damaged tissues in order to restore healthy function to muscles, tendons, ligaments and nerves that have been affected by inflammation, injuries or various traumas. The combination edge profile is formed by a double wide radius following the full thickness of the tool similarly as a spherically blunted tangent ogive, including a nose radius covering around 90 degrees of the curved edge; the double specific radius sharpness angle varying from 1 degree near the full tool thickness of the instrument up to around 50 degrees as an average, but reaching around 90 degrees just when it reaches the much smaller tip edge radius.

### REFERENCE TO PROVISIONAL

This application is a continuation of the provisional patent, application No. 61/425,248, filed Dec. [20] 21, 2010 entitled Soft Tissue Tech abbreviated ST2.

### BACKGROUND OF INVENTION

The majority of people suffer from neuro-musculo-skeletal pain sometime throughout their life. This pain is caused by either neurological compromise or by the soft tissue structures, such as the ligaments, tendons, muscles or fascia. Inflammation causes scar tissue, also known as fibrotic adhesions or fibrosis, to build up in and on the soft tissue structures mentioned above. Things like over training, not stretching before and after a workout, experiencing trauma to the soft tissue, or having a repetitive strain injury can cause fibrosis to line these structures and cause chronic pain.

Fascia lines every muscle in the body. This fascia is supposed to be mobile and not adhere to the muscle layer below it. After any of the above actions that cause inflammation to occur, the inflammatory process is activated. When this process is resolved, there are fibrotic adhesions left behind that adhere the fascia to the underlying muscle and tendon. This adherence causes decreased range of motion for the muscle and joint that it moves. This same concept of inflammation and fibrosis happens to ligaments.

Ligaments have a certain amount of plasticity which allows them to return to their normal length after they have been stretched when a joint reaches its end range or just beyond. When there are fibrotic adhesions on a ligament it loses its resilience and upon stretching the ligament doesn't return to its original length. This laxity in the ligament causes joint hypermobility, which in turn causes the muscles to spasm and secure the hypermobile joint. The increased or decreased motion in the joint causes degenerative changes to the cartilage. Also, the non resilient ligament may be easily torn.

Cartilage in the joints do not receive nutrients from blood like almost every other structure in the body. Joints receive nutrients from diffusion. Motion is needed for this process to be effective. So, if a joint is restricted, the joint degenerates. The same is true for too much motion, the cartilage breaks down quicker than it is repaired due to too much movement. Basically, the nerves, muscles, tendons and ligaments have to function properly in order for the joints to function properly.

In recent years, there have been many designs to treat soft tissue dysfunction. Many of these instruments have shapes to match body contours. Some designs have multiple tools to treat different areas. The majority of these tools are finished to a shiny/slippery surface. In addition to a slippery surface, the treatment edges are either a single bevel or a double bevel.

The following U.S. patents related to those designs are:

| | |
|---|---|
| John G. Louis. | No. 7,431,706 |
| Jung-Miin Lin. | No. 6,077,239 |
| David A. Graston. | No. 5,441,478 |
| Roman A. Wolocko. | No. 4,483,328 |
| Mark J. Scappaticci. | No. D634,022 |
| Ya-Ting Liang. | No. D524.445 |

Most treatment tools today have a single or double bevel edge with a taper or conical section which does not permit either double functions or double direction of treatment with the tool; furthermore, their straight taper or bevel edge sharpness is not conducive to a progressive breakdown of fibrous adhesions, scar tissue and trigger points since the only constant parameter of current instruments is the edge shape and its tip radius edge of 50 degrees or more, the practitioner is obliged to constantly vary the tool's angle position in relation of the tissue surface to be treated; therefore, the treatment success is highly related to the intuitive or learned practitioner skill.

### SUMMARY OF INVENTION

The invention, referring to the soft tissue tech instrument, also known as ST', is an instrument designed to break up fibrous adhesions, trigger points and scar tissue in any of the soft tissues mentioned above; the double radius edge is designed to allow the practitioner to treat in both directions without turning the instrument around, this double edge is actually a triple curved radius, similarly to a spherically blunted tangent ogive with a rounded edge profile can help pinpoint the treatment to the damaged area, improving the treatment, as well as making it more comfortable for the patient being treated.

The instrument has four distinct treatment edges which correspond to different contours of the body, it has one large convex treatment edge and one large concave treatment edge opposite the large convex edge, one medium convex treatment edge and one small convex treatment edge opposite the medium convex edge; the large concave treatment edge is designed to treat large tissues like the hamstrings, quads and torso, while the large convex treatment edge opposite the convex treatment edge treats the same tissues but more aggressively, the convex treatment edge pinpoints its treatment in a smaller area within the large tissue, the medium convex treatment edge is designed to treat small to medium tissues and medium to large joints like hands, feet, lower legs, ankles, knees, shoulders, elbows and wrists, the small convex treatment edge is designed to treat smaller tissues and those harder to get to areas like parts of the hands, fingers, the carpal tunnel and the supraspinatus tendon.

The instrument is made from a hard non-porous malleable material, this allows it to be sterilized, so not to transfer organisms from patient to patient, it has two roughened surfaces for gripping the instrument; when an emollient is applied to the skin of the patient, this lubricant is then transferred to the instrument upon treatment; once the tool is turned around to use an alternate treatment edge, it would be difficult to hold the instrument due to the now slippery surface, the roughened surface creates a surface that is easily gripped by the practitioner which minimizes fatigue of the practitioners hands, it has a double radius sharpness angle treatment edge varying from a very small angle leaving the full thickness of the tool up to an average of 50 degrees, but reaching 90 degrees just where it reaches the tip edge radius; this treatment edge is all the way around the instrument with a very small tip edge radius.

### OBJECTS & ADVANTAGES

The treatment edge of the instrument starts at the full thickness of the tool with an angle of sharpness 0-1 degrees with an average of around 50 degrees for the 2 radius coming from each side of the tool, and finally reaching over 90 degrees where those radius reach the tip radius edge of the tool; this is where this new tool differs greatly from prior art and existing tools on the market. This continuous change of sharpness angle on the edge profile is where the instrument progressively increases its force on the tissues to be displaced until the sharper angle provides the extra force to break down the adhesions and restore the normal elasticity of the tissue.

The ST² is designed for use by a practitioner for soft tissue mobilization and myofascial release. The instrument is used by applying pressure with the double beveled treatment edge, FIG. 3 numbers 2 and 3 to the skin, particularly a human. An emollient is applied to the surface of the skin in order to slide the instrument along the surface. This sliding of the instrument is in all directions along the surface of the skin with a purpose of breaking up fibrous adhesions between the fascial layer and either the muscular, tendonous or ligamentous layer. Fascia surrounds every muscle, ligament and tendon throughout the human body. Fibrous adhesions develop between the layers due to either acute trauma, a repetitive motion disorder or chronic inflammation. These adhesions cause chronic pain and decreased range of motion. Because of the instrument's thickness and hard material, the practitioner can feel where the adhesions or trigger points are located. It feels gritty underneath the instrument where there are fascial adhesions. This would not be as evident with the use of the practitioners hand The vibrations produced are being amplified through the instrument.

The ST² is used to break up these fibrous adhesions. In the process of treatment, it also causes a controlled inflammation, which brings in blood flow and rinses away the fibrous adhesions the practitioner just broke up while providing nutrients for healing.

The ST² is also used for trigger point therapy, as well as scar tissue removal and reorganization. It's treatment edges ultimately break up the taut bands that make up a trigger point. It also causes a controlled inflammation, which brings in nutrients, mainly ATP allowing for the release of the actin heads from the myosin filaments. This causes the taut bands to release, thus elimination of the trigger point. The ST² helps break up scar tissue formation by the same method as breaking up fibrous adhesions. This is needed in cases where a patient is injured and tears a ligament or tendon. This tear is healed with scar formation. The problem is the scar tissue is laid down in an unorganized fashion, which restricts its full strength, range of motion and functionality. The ST² instrument can help break up some of the scar tissue. Immediately after treatment, the patient will stretch the ligament or tendon in order for the tissue to regain its original range of motion. This process is repeated until the tissue is fully healed and range of motion has been restored.

The soft tissue tech has 4 treatment edges specifically contoured for the multitude of body contours. The double beveled treatment edge allows the practitioner to treat in all different directions without having to turn the instrument around. This allows for more fluid movements in comparison to a single bevel. The large concave treatment edge is for working less aggressively with medium to large tissues like legs, arms, hips, chest and back. The large convex treatment edge is for working more aggressively with medium to large tissues like legs, arms, hips, chest and back, seeing as it is opposite the surface being treated. The medium convex treatment edge is for working with small to medium tissues and medium to large joints like knees, shoulders, elbows, ankles, wrists, ankles, hands and feet. The small convex treatment edge is for working with smaller tissues and those harder to get to areas like the supraspinatus tendon, carpal tunnel, hands and feet.

The treatment edge, which is a spherically blunted tangent ogive, pinpoints the treatment even further than just one radius with a flat bevel; furthermore, this treatment edge progressively breaks down scar tissue and fibrous adhesions more effectively and comfortably for the patient. There is an evident correlation between several science fields such that fibrous adhesions, trigger points and traumatized tissues react to therapeutic treatment following similar principles such as fluid motion, hydrodynamic and granular particle displacement, this is why the new therapeutic instrument is formed with a specific edge profile. Reference; “Nose Cone Design” by www.wikipedia.org, and “The Descriptive Geometry of Nose Cones” by Gary A. Crowell Sr.

### BRIEF DESCRIPTION OF THE DRAWINGS

This invention's objects, features and advantages will be better understood in consideration of the detailed description of several embodiments illustrated in the drawings, in which:

FIG. 1 is a top view of the first embodiment of the ST2 Instrument.

FIG. 2 is a sectional view of FIG. 1 following line 1-1.

FIG. 3 is a top view of a second embodiment of the ST2 Instrument.

FIG. 4 is a cut-off view of FIG. 3 following line 2-2.

### LIST OF REFERENCE NUMERICALS

10 elongated ST2 tool.

12 textured surface

13 large radius bevel

14 rounded edge

15 large radius end

16 large concave edge

17 large convex edge

18 small radius end

20 compact ST2 tool

22 textured surface

23 large radius bevel

24 rounded edge

25 large radius end

26 concave edge

27 convex edge

28 small radius end

29 straight edge section

### DETAILED DESCRIPTION OF THE INVENTION

The description of the ST2 tools is related to the shape of two soft tissue tech therapeutic tools or instrument, an elongated and a compact one as a combination set.

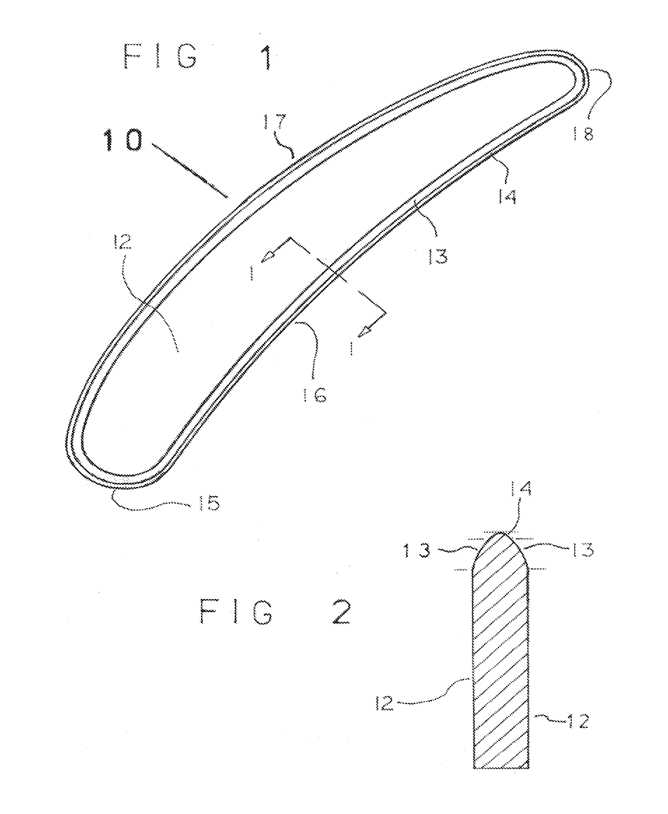

FIG. 1 and 2 top and sectional views of an ST2 tool illustrates a curved elongate instrument of dimensions of around 9 inches long by 1.5 inches wide with a thickness of around 0.2 inches; the tool 10 has a texture finish 12 on both sides is a graspable surface; this surface is followed by a large radius (around ¼″ radius) double bevel 13 with a rounded tip edge 14 of around 0.030 inch of radius; the tools outer shape includes a wide concave edge 14, a long convex edge 17 linked by a medium radius end in 15 and a smaller radius end in 18.

The FIG. 2 cross-sectional view illustrated how the triple radius bevel edge is shaped in the form of a tangent ogive ending with a small radius nose, where the radius of the large bevel is around or wider than the thickness of the tool.

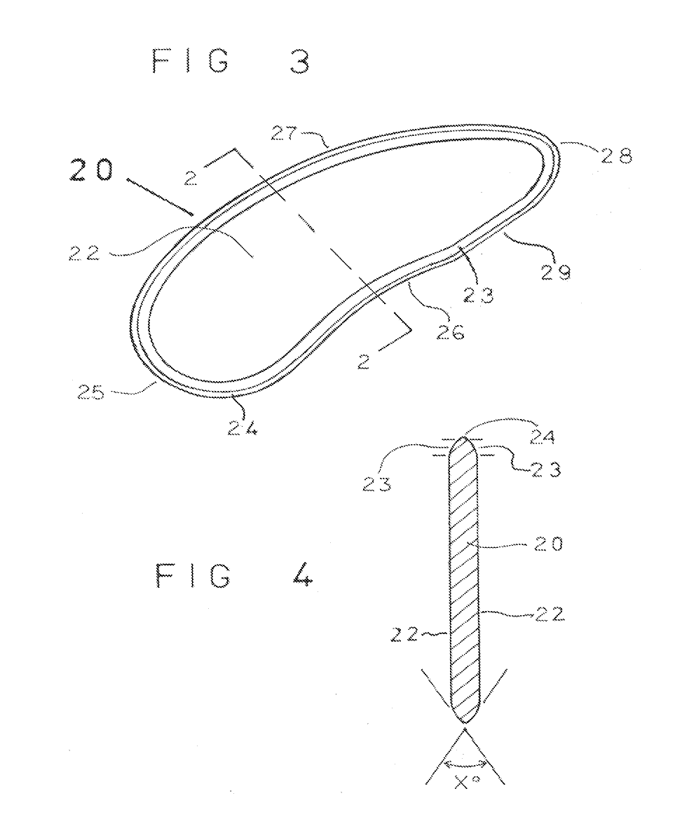

FIGS. 3 and 4 illustrate the top and cross-sectional views of the compact ST2 tool, this second embodiment of the instrument is shorter but wider than the first embodiment with length of around 6.5 inches, width of around 2.5 inches and similar thickness.

FIG. 3 instrument 20 is illustrated by a texture, top and bottom surface 22, this surface is followed by a large radius (around ¼ inch) double radius surface 23 with a joining rounded tip edge 24 of around 0.030 inch of radius; the tools outer shape includes a large radius end 25 followed on each side by a concave edge 26 and a wide convex edge 27, the second end shorter radius 28 is followed by a straight section of around 1.5 inches in length.

FIG. 4 illustrates the cross-sectional view of the instrument 20 with its double texture surface 22 and treatment edge triple radius bevel 23-24-23 which formed an approximate angle X degrees of around 50 degrees of sharpness.

It is to be noted that the sharpness angle of the tool edge very progressively from 0-1 degrees when leaving the full thickness of the tool reaching up to 90 degrees just before the small radius tip edge, therefore the average sharpness is taken by the straight line starting at the start of the ogive radius and going to the start of the small radius forming the tip of the edge; this average generally falls between 45 and 60 degrees.

The fabrication of the ST2 instrument follows the same process of a similar part done with the same material, as an example, the first choice would be stainless steel which would start as a steel plate or bar of the right thickness in a low or medium state of hardness;

machining of the specific radius and smaller tip radius can be done with a specialized, double radius end mill done on each side of the plate which had been cut first at the desired shape; the machining of the specific radius and smaller tip radius can also be done by a punch press with two dyes to cut the treatment edge.

A more elaborate process would be to cut both sides at the same time with a cutter having the triple radius profile of the edge.

Another process could be powder metallurgy forming where high density up to 99.7 percent can be achieved including a sintered high quality finish.

The slip surface is easily achieved through sand blasting the graspable surface while the edge is protected with an appropriate tape.

Although the said invention is meant for use on human tissue, that is not to say it cannot be used to treat animals; it can be used on any muscle, tendon, ligament or fascial lining of any mammal; in addition, the invention is meant for use by a qualified practitioner, but may be used by a patient for in-home use with the proper instruction; the many advantages which are inherent to the embodiment structure are obvious to the one skilled in the art. Those embodiments are described herein illustratively and are not meant to limit the scope of the invention, therefore variations of the basic embodiment are intended to be encompassed by the following claims.

The invention claimed is:

1. A soft tissue therapeutic tool comprising:
a. a graspable rigid elongated tool body providing a curved long positive edge opposite to a curved negative edge joined at each end by two smaller curvature radius;
b. the tool body having a thickness of substantially 0.2 inch with graspable surface having a texture finish adapted to provide a grip surface for the hand;
c. the tool body having an edge with a tapered profile at the tool body's periphery including one specific radius from each side of the tool joined at a tip edge by a radius of substantially 1/50 inch; and
d. the tool body being formed out of a material selected from a metal, an alloy material, and a composite that has suitable qualities of finish, hardness, sanitariness, and resonance.

2. The soft tissue therapeutic tool of claim 1 wherein the dimension of the specific tapered profile radius is substantially 0.3 inch.

3. The soft tissue therapeutic tool of claim 2 wherein the edge profile has a sharpness angle of substantially 50 degrees.

4. The soft tissue therapeutic tool of claim 3 wherein the tool body has a spherical tip that forms a segment of circle of substantially 80 degrees.

5. The soft tissue therapeutic tool of claim 1 wherein the tool body comprises a combination triple radius profile edge which forms a spherically blunted tangent ogive in which the ogive radius is around 1.5 times the thickness of the tool body and a blunted nose segment thereof has radius that is substantially 0.3 times the tool body thickness.

6. The soft tissue therapeutic tool of claim 4 where the tool length and width are substantially 9 inches by 1.5 inches respectively, and the two ends of the tool each have a different radius.

7. The soft tissue therapeutic tool of claim 4 wherein the specific edge profile provides a superior treatment effectiveness because the edge profile is adapted to displace the tissue in a more physiological correct manner.

8. A soft tissue therapeutic tool comprising:
a. a graspable rigid elongated tool body having a curved positive edge opposite to a curved negative edge joined to each other by two shorter-radius curved ends;
b. the tool body having a thickness of substantially 0.2 inches with graspable surface having a texture finish adapted to provide a suitable grip for the hand;
c. the tool body having a tapered profile edge at the tool body's periphery including one specific radius from each respective side of the tool body, and joined at a tip edge thereof having a radius of substantially 1/32 inch; and
d. said tool body being formed out of a material selected from a metal, an alloy material, or a composite that has suitable qualities of finish, hardness, sanitary, and resonance

9. The soft tissue therapeutic tool of claim 8 wherein the said one specific radius is substantially 0.25 inch.

10. The soft tissue therapeutic tool of claim 9 wherein the said tool body has a general shape of is similar to a kidney bean with a larger radius at one end and a smaller radius at the other end.

11. The soft tissue therapeutic tool of claim 10 wherein including double wide radius edge profile with a sharpness angle of substantially 50 degrees.

12. The soft tissue therapeutic tool of claim 11 wherein the tool body has a spherical tip that forms a segment of circle of substantially 80 degrees.

13. The soft tissue therapeutic tool of claim 8 wherein the tool body comprises a combination triple radius profile edge which forms a spherically blunted tangent ogive having an ogive radius substantially 1.5 times the thickness of the tool body and having a blunted nose segment radius that is substantially 0.3 times the tool thickness.

14. The soft tissue therapeutic tool of claim 12 where the tool body has a length and a width of substantially 6 inches and 2 inches, respectively, and two ends of the tool are each of different radius; with one curved negative edge at a junction to the smaller radius end providing a straight edge of substantially 1.5 inches while maintaining the respective shapes of the concave edge and the smaller-radius end.

15. The soft tissue therapeutic tool of claim 13 wherein the edge profile is adapted to provide superior treatment effectiveness in that the edge profile displaces the tissue in a more physiologically correct manner.

16. A soft tissue therapeutic tool comprising:
a. a graspable rigid mini tool having one curved positive edge opposite to a curved negative edge joined to each other by two shorter-radius curved ends;
b. the mini tool having a predetermined thickness of substantially ⅛ inch and having a graspable surface which is texture finished and adapted to provide the a satisfactory grip for the hand;
c. the mini tool having a tapered profile edge at the mini tool's periphery the profile edge having a specific radius from each side of the mini tool joined at the a tip edge the latter having a radius of around 0.02 inch; and
d. the tool being formed out of a material selected from metal, an alloy material, or a composite that has suitable qualities of finish, hardness, sanitariness, and resonance.

17. The soft tissue therapeutic tool of claim 16 wherein the general shape of the mini tool is similar to an oblong rectangular kidney bean with a wide convex side opposite to a wide concave side with each ends of smaller double radius corner for a total dimension of substantially 4 inches by 2.5 inches.

18. The soft tissue therapeutic tool of claim 1 wherein the preferred material is stainless steel.

19. The soft tissue therapeutic tool of claim 8 wherein the preferred material is stainless steel.

20. The soft tissue therapeutic tool of claim 16 wherein the preferred material is stainless steel.
